(19) United States
(12) Patent Application Publication
(10) Pub. No.: US 2004/0009185 A1
(43) Pub. Date: Jan. 15, 2004

(54) Enhancing the immune response to an antigen by presensitzing with an inducing agent prior to immunizing with the agent and the antigen

(76) Inventors: Peter Emtage, Boston, MA (US); Brian H. Barber, Mississauga, CA (US); Suryprakash Sambhara, Decatur, GA (US); Charles Dwo Yuan Sia, Toronto (CA)

Correspondence Address: Patrick J Halloran, Aventis Pasteur Inc, Knerr Building, One Discovery Drive, Swiftwater, PA, 18370, US

(21) Appl. No.: 10/168,417

(22) PCT Filed: Jan. 5, 2001
(86) PCT No.: PCT/CA01/00005

(51) Int. Cl. A61K 39/02; A61K 39/08; A61K 39/00

(52) U.S. Cl. 424/185.1; 424/239.1; 424/236.1

(57) ABSTRACT

A method of enhancing an immune response is disclosed. Th method involves an initial priming of the animal with an inducing agent, subsequently followed by administration of an inducing agent-antigen mixture. The antigen may be a tumour associated antigen, pathogenic organism antigen, autoimmune antigen, immunogenic fragment thereof, or a nucleic acid coding therefor.

### FIELD OF INVENTION

The present invention relates to methods and compositions for enhancing an immune response to an antigen in an animal.

### BACKGROUND TO THE INVENTION

Vaccines have been used with a high rate of efficiency to prevent infectious diseases caused by agents as diverse as bacteria, viruses and parasites (Plotkin, S. A. and Orenstein, W. A. (eds.), Vaccine, 3rd ed., W. B. Saunders, Philadelphia, U.S.A. (1991)). Furthermore, a diverse array of immunopotentiating and/or adjuvant-like materials have also been co-administered with said vaccines to augment the immune response (Gupta, R. K. and Siber, G. R., Vaccine 13:1263-1276 (1995); Cox, J. R. and Coulter, A. R., Vaccine 15:248-256 (1997); Plotkin, S. A. and Orenstein, W. A., supra, pp. 36-37):

A number of bacterial toxins have demonstrated immunopotentiating characteristics. These include Staphylococcal toxins (Koppler, J. et al., Science 224:811-817 (1989); White, J. et al., Cell 56:27-35 (1989); WO 98/26747; EP 839536; U.S. Pat. No. 5,182,109), Escherichia coli toxins (Dickinson B. L. and Clements, J. D., Infect. Immun. 63:1617-1623 (1995); Douce, G. et al., Proc. Natl. Acad. Sci. 92:1644-1648 (1995); U.S. Pat. No. 5,182,109) and Streptococcal, Mycoplasma arthritidal, and/or Yersinia enterocolitical toxins (WO 98/26747).

Additionally, the modification of an antigen in controlled manner has also been demonstrated to enhance immunogenicity for that antigen. For example, carrier proteins (e.g. tetanus toxoid (TT), diphtheria toxoid (DT)), when coupled to T-independent antigens, haptens or weak immunogens enhance the immunogenicity of the antigens coupled to these proteins (Herrington, D. A. et al., Nature 328: 257-259 (1987); Nash, H. et al., Fertil. Steril. 34: 328-335 (1980); Robbins, J. B. and Schneerson, R., J. Infect. Dis. 161:821-832 (1990); Powell M. F. and Newman, M. J. (eds.), Vaccine Designs—The Subunit and Adjuvant Approach, Plenum Publishing Corp., New York, N.Y., U.S. A. (1995)).

Specifically, tetanus toxoid absorbed with aluminum salts and with preservatives such as Thimerosal (Trademark) given alon or in combination with other bacterial antigens has been us d not only as a vaccine to prevent neonatal or adult tetanus (e.g. Plotkin, S. A. and Orenstein, W. A., supra, Chpt. 18, pp. 441-474), but also as an agent to induce enhanced humoral immune responses against bacterial toxins/subunits or viral antigens when coupled as a carrier molecule thereto and/or when co-administered with the vaccine/immunogen to which an immune response is desired (for example, Herrington, D. A. et al., Nature 328:257-259 (1987); Nash, H. et al., Fertil. Steril. 34:328-335 (1980); Robbins, J. B. and Schneerson, R., J. Infect. Dis. 161:821-832 (1990); Kaistha, J. et al., Indian J. Pathol. Microbiol. 39: 287-292 (1996); Mukerjee, R. and Chaturvedi, U. V. C., Clin. Exp. Immunol. 102:496-500 (1995); U.S. Pat. Nos. 4,673,574, 4,751,064, 5,877,298).

In the context of Haemophilus influenzae related conjugate vaccines utilizing tetanus toxoid or diphtheria toxoid as carrier, it has been observed that the humoral immune response to the conjugated immunogen is augmented after immune priming to the carrier (Granoff, D. M. et al., J. Pediatr. 121: 187-194 (1992); Granoff, D. M. et al., Pediatr. Res. 85: 694-697 (1993). In contrast, Ferro and Stimson (Drug Design and Discovery 14:179-195 (1996)) have demonstrated that animals presensitized with tetanus toxoid exhibit a significantly lower antibody response to a tetanus toxoid conjugated immunogen (gonadotrophin releasing hormone (GnRH)—tetanus toxoid) by comparison to immunization with conjugated immunogen in the absence of tetanus toxoid presensitization.

In view of the foregoing, there is a need in the art to develop improved vaccination protocols and compositions that enhance the immune response to an antigen in the vaccine.

### SUMMARY OF THE INVENTION

The present inventors have determined that the immune response to an antigen can be greatly improved or enhanced if the animal is first primed with a foreign protein or inducing agent and then receives the antigen in admixture with the inducing agent. The immune respons generated using such a protocol is enhanced several fold ov r when the antigen alone, without the inducer, is used. The method is advantageous as it provides the enhancement or augmentation of the immune response to an antigen and/or improves a vaccination protocol by allowing one to use less antigen.

Accordingly, the present invention provides a method of enhancing an immune response to an antigen in an animal comprising (a) administering an inducing agent to the animal followed by (b) administering the inducing agent and the antigen to the animal.

In one embodiment of the invention, the inducing agent is a bacterial toxoid such as tetanus toxoid or diphtheria toxoid.

The antigen can be any antigen. In one embodiment, the antigen is selected from the group consisting of tumour antigens, pathogenic organism antigens, autoimmune antigens, and immunogenic fragments thereof.

The antigen and/or inducing agent may be administered directly or the nucleic acid encoding the antigen and/or inducing agent may be employed. In the latter case, the nucleic acid coding for the antigen and/or inducing agent may be in a vector, plasmid, bacterial DNA or may be naked/free DNA or RNA.

In yet additional aspects of the invention, the antigen and inducing agent may additionally be administered in conjunction with at least one member selected from the group consisting of cytokines, lymphokines, co-stimulatory molecules and nucleic acids coding therefor, and adjuvants.

The invention also includes vaccine compositions comprising an antigen and an inducing agent in admixture with a pharmaceutically acceptable diluent or carrier.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become appar nt to thos skilled in the art from this detailed description.

### BRIEF DESCRIPTION OF THE DRAWINGS

The invention will now be described in relation to the drawings in which:

FIG. 1 (and SEQ.ID.NO.:1) shows the nucleic acid sequence of modified gp100.

FIG. 2 (and SEQ.ID.NO.:2) shows the amino acid sequence of modified gp100.

FIG. 3 (and SEQ.ID.NO.:3 and 4) shows the nucleic acid and amino acid sequence of a modified CEA.

FIG. 4 is a bar graph demonstrating the effect of tetanus toxoid priming on the immunogenicity of recombinant ALVAC (2) vectors expressing a modified gp100 gene in A2Kb transgenic mice.

FIG. 5 is a bar graph demonstrating the effect of tetanus toxoid priming on the immunogenicity of recombinant ALVAC (1) vectors expressing a modified gp100 gene in A2Kb transgenic mice.

FIG. 6 is a bar graph demonstrating the effect of tetanus toxoid priming on the immunogenicity of recombinant ALVAC vectors expressing CEA in A2Kb transgenic mice.

FIG. 7 is a bar graph demonstrating the effect of tetanus toxoid and diphtheria toxoid priming on the immunogenicity of recombinanty ALVAC vectors expressing a modified gp100 gene in A2Kb transgenic mice.

FIG. 8 is a bar graph demonstrating the effect of tetanus toxoid priming on the immunogenicity of recombinant ALVAC vectors expressing native or modified gp100 in A2Kb transgenic mice.

### DETAILED DESCRIPTION OF THE INVENTION

As hereinbefore mentioned, the present inventors have developed an improved vaccination protocol wherein the immune response to an antigen is enhanced if the animal is first primed with an inducing agent and then subsequently receives the antigen in admixture with the inducing agent.

Accordingly, the present invention provides a method of enhancing an immune response to antigen in an animal comprising (a) administering an effective amount of an inducing agent to the animal (sometimes referred to as step (a) hereinafter) followed by (b) administering an effective amount of the inducing agent and the antigen (sometimes referred to as st p (b) hereinafter) to the animal.

The term “animal” as used herein includes all members of the animal kingdom including mammals, preferably humans.

The term “enhancing an immune response” is defined as enhancing, improving or augmenting any response of the immune system, for example, of either a humoral or cell-mediated nature. The enhancement of an immune response can be assessed using assays known to those skilled in the art including, but not limited to, antibody assays (for example ELISA assays), antigen specific cytotoxicity assays and the production of cytokines (for example ELISPOT assays). Preferably, the method of the present invention enhances a cellular immune response, more preferably a cytotoxic T cell response.

The term “effective amount” of the inducing agent or the inducing agent and the antigen means an amount effective, at dosages and for periods of time necessary to enhance an immune response.

The term “inducing agent” as used herein means any agent that when used in the method of the invention can enhance, augment or improve an immune response to an antigen. For example, the inducing agent enhances an immune response as the immune response to the antigen is greater when the inducing agent is administered in both steps (a) and (b) of the method of the invention than when the antigen alone is administered. The method of the invention may also be used to improve an immune response as in the presence of an inducing agent one can generally administer a lower concentration of the antigen than when the inducing agent is not used and still generate a comparable or perhaps enhanced immune response.

The inducing agent can either be an agent to which the recipient animal is naive or to which the recipient animal has been previously exposed. The inducing agent is preferably a foreign or non-self protein. Suitable proteins include, but are not limited to, natural peptides and proteins, (such as bovine serum albumin) including proteins derived from bact rial, viral, parasitic, fungal, mycosal and mammalian sources. In one embodiment, the protein inducing agent is a bacterial toxoid derived from a bacterial toxin by their synthetic, chemical, physiochemical or genetic modification (e.g. Diphtheria toxoid, CRM197, Tetanus toxoid, Pertussis toxoid, Pseudomonas aeruginosa recombinant exoprotein A and Clostridium perfringens exotoxins). Other proteins derived from bacteria may also be employed. The bacterial source may be, for example, Haemophilus influenzae, Meningococci, Pneumococci, β-hemolytic streptococci, E. coli, Vibrio, Salmonella, Staphylococci, Helicobacter and Campylobacter. Viral sources include influenza HA, NA or RSV capsid proteins.

The term “antigen” as used herein means any agent to which one wishes to generate an immune response.

Antigens are usually proteins, but may belong to other classes of macromolecules, such as carbohydrates and the like. Protein antigens include both self antigens, such as tumor antigens and autoimmune antigens as well as non self antigens such as antigens derived from pathogenic organisms including viruses, bacteria, fungi, parasites, protozoans and yeast. Antigens may be obtained from natural sources or from host cells genetically engineered to produce the antigens.

The term “administering” is defined as any conventional route for administering an antigen to an animal for use in the vaccine field as is known to one skilled in the art. This may include, for example, administration via the parenteral (i.e. subcutaneous, intradermal, intramuscular, etc.) or mucosal surface route. The antigen and inducing agent may also be administer d directly to a lymphatic site for example directly into a lymph node. The initial step of the method of the invention, i.e. step (a) administering the inducing agent to the animal, may be generally referred to as “pre-priming”. The pre-priming of an animal can be achieved in a single dose or repeated at intervals. As such, the dose of the inducing agent may vary according to factors such as the health, age, weight and sex of the animal. The dosage regime may be adjusted to provid the optimum induction of the immune response. On skilled in the art will appreciate that the dosage regime can be determined and/or optimized without undue experimentation.

The inducing agent and the antigen may be administered in various forms and combinations. For example, when either the inducing agent and/or the antigen is a protein they may be administered in the form of the protein or as a nucleic acid encoding the protein. Therefore, when either the inducing agent and/or the antigen is a protein the term “administering an inducing agent” or “administering an antigen” includes both the administration of the protein and the administration of the nucleic acid encoding the protein. When both the inducing agent and antigen are proteins they may be each administered as proteins, each administered as nucleic acids encoding the protein or one may be administered as a protein and the other as a nucleic acid encoding the protein as well as various combinations or permutations of these.

In one example, the inducing agent may be administered as a protein in both step (a) and step (b) of the method of the invention while the antigen may be administered as a nucleic acid encoding the antigen. In a further example, the inducing agent may be administered as a nucleic acid in both step (a) and step (b) and the antigen can be administered as a protein. In another example, the inducing agent may be administered as either a protein or a nucleic acid in step (a) and as a nucleic acid in step (b) and the antigen can be administered as a nucleic acid. In such an embodiment, the inducing agent and the antigen may be prepared as a chimeric nucleic acid sequence comprising a first nucleic acid sequence encoding an inducing agent linked to a second nucleic acid sequence encoding the antigen. As such, upon administration of the chimeric nucleic acid sequence to the animal, the inducing agent and the antigen will be expressed in vivo as a recombinant fusion protein. In another example, the inducing agent may be administered as either a protein or a nucleic acid in step (a) and as a protein in step (b) and the antigen may be administered as a protein. In such an embodiment, the inducing agent and antigen may be covalently linked for example they may be prepared as a recombinant fusion protein in vitro or they may be linked by other means including chemical crosslinking as described e.g., in U.S. Pat. No. 5,153,312. There are several hundred crosslinkers available that can conjugate two proteins. (See for example “Chemistry of Protein Conjugation and Crosslinking”. 1991, Shans Wong, CRC Press, Ann Arbor). The crosslinker is generally chosen based on the reactive functional groups available or inserted on the ligand. In addition, if there are no reactive groups a photoactivatible crosslinker can be used. In certain instances, it may be desirable to include a spacer between the ligand and the oil-body protein. Crosslinking agents known to the art include the homobifunctional agents: glutaraldehyde, dimethyladipimidate and Bis(diazobenzidine) and the heterobifunctional agents: m-Maleimidobenzoyl-N-Hydroxysuccinimide and Sulfo-m Maleimidobenzoyl-N-Hydroxysuccinimide.

In one embodiment of the invention, tetanus toxoid is used as an inducing agent. In another embodiment of the invention, diphtheria toxoid is used as an inducing agent. The tetanus toxoid or diphtheria toxoid may b prepared by methodologies well known to those skilled in the art and are commercially available from Aventis Pasteur, Smithkline Beecham, Lederle, Statens Inst. etc. Generally, the production of the toxoid can be divided into 5 stages, namely maintenance of the working seed, mass growth from the working seed, harvest of the toxin, detoxification of the toxin, and purification of the toxoid (for example, as set out in U.S. Pat. No. 5,877,298, which is incorporated herein by reference). As contemplated by this invention, tetanus toxoid or diphtheria toxoid is used as such, or can be further adsorbed with aluminum salts and/or admixed with preservatives such as Thimerosal (Trademark), or formulated in additional ways as will be known to those skilled in the art.

In embodiments of the invention employing antigens that are relatively small polypeptides, the antigen may be synthesized in vitro using techniques well known to the person skilled in the art. By “polypeptide” or “protein” is meant any chain of amino acid, regardless of length or post-translational modification (e.g., glycosylation or phosphorylation). Both terms are used interchangeably in the pres nt application. The terms “polypeptide”, or “protein” as used herein are also intended to include analogs of antigens containing one or more amino acid substitutions, insertions and/or deletions. Amino acid substitutions may be of a conserved or non-conserved nature. Conserved amino acid substitutions involve replacing one or more amino acids with amino acids of similar charge, size, and/or hydrophobicity characteristics. Non-conserved substitutions involve replacing one or more amino acids with one or more amino acids which possess dissimilar charge, size, and/or hydrophobicity characteristics. Amino acid insertions may consist of single amino acid residues or sequential amino acids. Deletions may consist of the removal of one or more amino acids or discrete portions of the polypeptide/protein. The deleted amino acids may or may not be contiguous.

As previously noted, one category of antigen is an antigen from a pathogenic organism. Various peptides have been found to be significant in stimulating a protective immune response in infectious diseases. Immunotherapeutic antigens useful for the treatment of infectious diseases may be obtained from pathogenic bacteria, viruses, and eukaryotes. For example, hepatitis viral peptides, HIV envelope peptides and plasmodium yoeli circumsporozoite peptide are capable of protecting the host against challenge with the infectious agent.

In other preferred embodiments, the antigen is a tumor antigen. The term “tumor antigen” as used herein includes both tumor associated antigens (TAAs) and tumor specific antigens (TSAs). A tumor associated antigen means an antigen that is expressed on the surface of a tumor cell in high r amounts than is observed on normal cells or an antigen that is expressed on normal cells during fetal development. A tumor specific antigen is an antigen that is unique to tumor cells and is not expressed on normal cells. The term tumor antigen includes TAAs or TSAs that have been already identified and those that have yet to be identified and includes fragments, epitopes and any and all modifications to the tumor antigens.

The tumor associated antigen can be any tumor associated antigen including, but not limited to, gp100 (Kawakami et al., J. Immunol. 154:3961-3968 (1995); Cox t al., Science, 264:716-719 (1994)), MART—1/Melan A (Kawakami et al., J. Exp. Med., 180:347-352 (1994); Castelli et al., J. Exp. Med., 181:363-368 (1995)), gp75 (TRP-1) (Wang et al., J. Exp. Med., 186:1131-1140 (1996)), and Tyrosinase (Wolfel et al., Eur. J. Immunol., 24:759-764 (1994); Topalian et at., J. Exp. Med., 183:1965-1971 (1996)); melanoma proteoglycan (Hellstrom et al., J. Immunol., 130:1467-1472 (1983); Ross et al., Arch. Biochem Biophys., 225:370-383 (1983)); tumor-specific, widely shared antigens, for example: antigens of MAGE family, for example, MAGE-1, 2, 3, 4, 6, and 12 (Van der Bruggen et al., Science, 254:1643-1647 (1991); Rogner et al., Genomics, 29:729-731 (1995)), antigens of BAGE family (Boel et al., Immunity, 2:167-175 (1995)), antigens of GAGE family, for example, GAGE-1, 2 (Van den Eynde et al., J. Exp. Med., 182:689-698 (1995)), antigens of RAGE family, for example, RAGE-1 (Gaugler et at., Immunogenetics, 44:323-330 (1996)), N-acetylglucosaminyltransferase-V (Guilloux et at., J. Exp. Med., 183:1173-1183 (1996)), and p15 (Robbins et al., J. Immunol.154:5944-5950 (1995)); tumor specific mutated antigens; mutated β-catenin (Robbins et al., J. Exp. Med., 183:1185-1192 (1996)), mutat d MUM-1 (Coulie et al., Proc. Natl. Acad. Sci. USA, 92:7976-7980 (1995)). and mutated cyclin dependent kinases-4 (CDK4) (Wolfel et al., Science, 269:1281-1284 (1995)); mutated oncogene products: p21 ras (Fossum et at., Int. J. Cancer, 56:40-45 (1994)), BCR-abl (Bocchia et al., Blood, 85:2680-2684 (1995)), p53 (Theobald et al., Proc. Natl. Acad. Sci. USA, 92:11993-11997 (1995)), and p185 HER2/neu (Fisk et al., J. Exp. Med., 181:2109-2117 (1995)); Peoples et al., Proc. Nat. Aced. Sci., USA, 92:432-436 (1995)); mutated epidermal growth factor receptor (EGFR) (Fujimoto et al., Eur. J. Gynecol. Oncol., 16:40-47 (1995)); Harris et al., Breast Cancer Res. Treat, 29:1-2 (1994)); carcinoembryonic antigens (CEA) (Kwong et al., J. Natl. Cancer Inst., 85:982-990 (1995)); carcinoma associated mutated mucins, for example, MUC-1 gene products (Jerome et al., J. Immunol., 151:1654-1662 (1993), loannides et al., J. Immunol., 151:3693-3703 (1993), Takahashi et al., J. Immunol., 153:2102-2109 (1994)); EBNA gene products of EBV, for example, EBNA-1 gene product (Rickinson et al., Cancer Surveys, 13:53-80 (1992)); E7, E6 proteins of human papillomavirus (Ressing et al., J. Immunol, 154:5934-5943 (1995)); prostate sp cific antig ns (PSA) (Xue et al., The Prostate, 30:73-78 (1997)); prostate specific membrane antigen (PSMA) (Israeli, et al., Cancer Res., 54:1807-1811 (1994)); PCTA-1 (Sue et al., Proc. Natl. Acad. Sci. USA, 93:7252-7257 (1996)); idiotypic epitopes or antigens, for example, immunoglobulin idiotypes or T cell receptor idiotypes, (Chen et al., J. Immunol., 153:4775-4787 (1994); Syrengelas et al., Nat. Med., 2:1038-1040 (1996)); KSA (U.S. Pat. No. 5,348,887); NY-ESO-1 (WO 98/14464).

Also included are modified tumor antigens and/or epitope/peptides derived therefrom (both unmodified and modified). Examples include, but are not limited to, modified and unmodified epitope/peptides derived from gp100 (WO 98/02598; WO 95/29193; WO 97/34613; WO 98/33810; CEA (WO 99/19478; S. Zaremba et al. (1997) Cancer Research 57:4570-7; K. T. Tsang et al. (1995) J. Int. Cancer Inst. 87:982-90); MART-1 (WO 98/58951, WO 98/02538; D. Valmeri et al. (2000) J. Immunol. 164:1125-31); p53 (M. Eura t al. (2000) Clinical Cancer Research 6:979-86); TRP-1 and TRP-2 (WO 97/29195); tyrosinase (WO 96/21734; WO 97/11669; WO 97/34613; WO 98/33810; WO 95/23234; WO 97/26535); KSA (WO 97/15597); PSA (WO 96/40754); NY-ESO 1 (WO 99/18206); HER2/neu (U.S. Pat. No. 5,869,445); MAGE family related (L. Heidecker et al. (2000) J. Immunol. 164:6041-5; WO 95/04542; WO 95/25530; WO 95/25739; WO 96/26214; WO 97/31017; WO 98/10780).

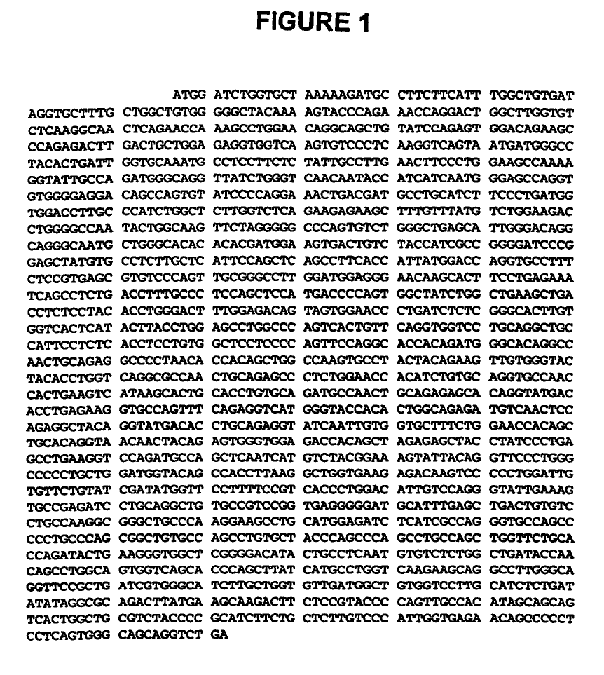

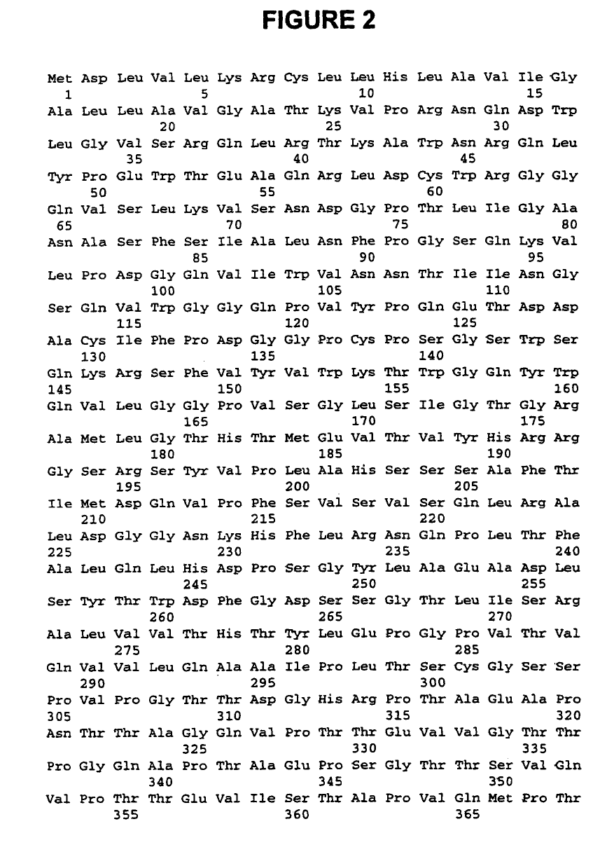

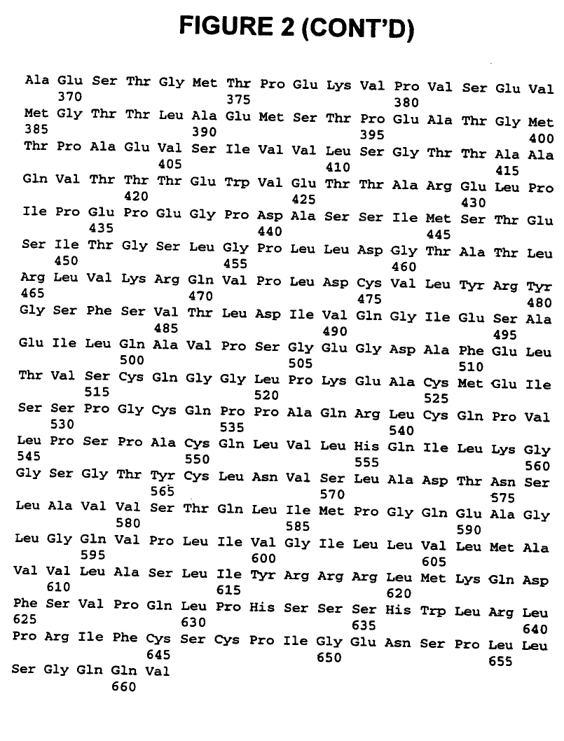

In a specific embodiment, the tumor-associated antigen is gp100, a modified gp100 or a fragment thereof. In one embodiment, the antigen is native gp100, the sequence of which is known in the art or a modified gp100 having a nucleic acid sequence shown in FIG. 1 and SEQ.ID.NO.:1 or an amino acid sequence shown in FIG. 2 or SEQ.ID.NO.:2. The modified gp100 antigen contains two mutations over the native gp100, at position 210 the threonine was replaced by methionine and at position 288 the alanine was replaced by valene. The modified gp100 is more fully described in U.S. application Ser. No. 09/693,755, filed on Oct. 20, 2000, which is incorporated herein by reference.

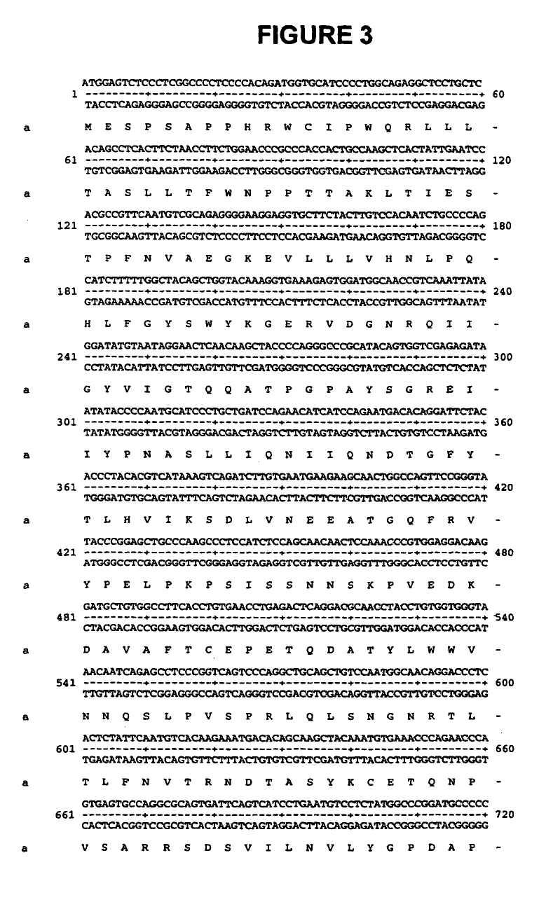

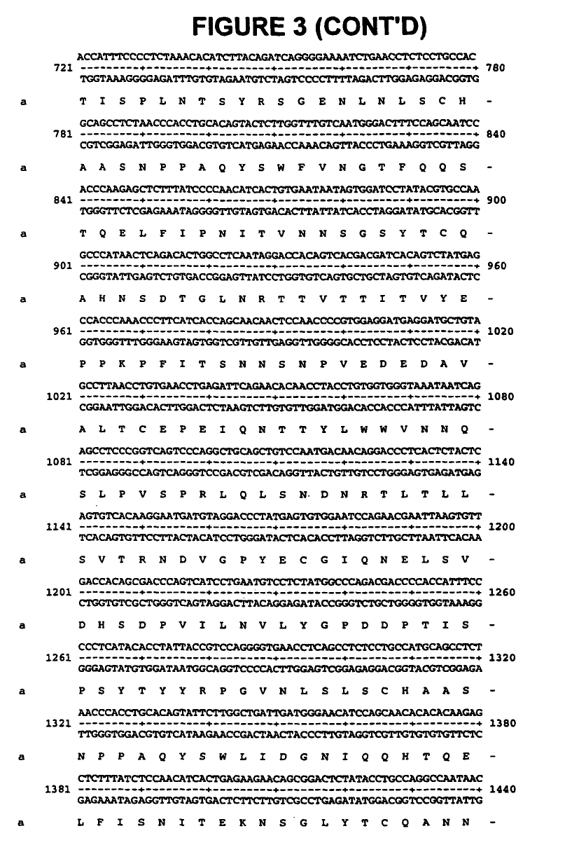

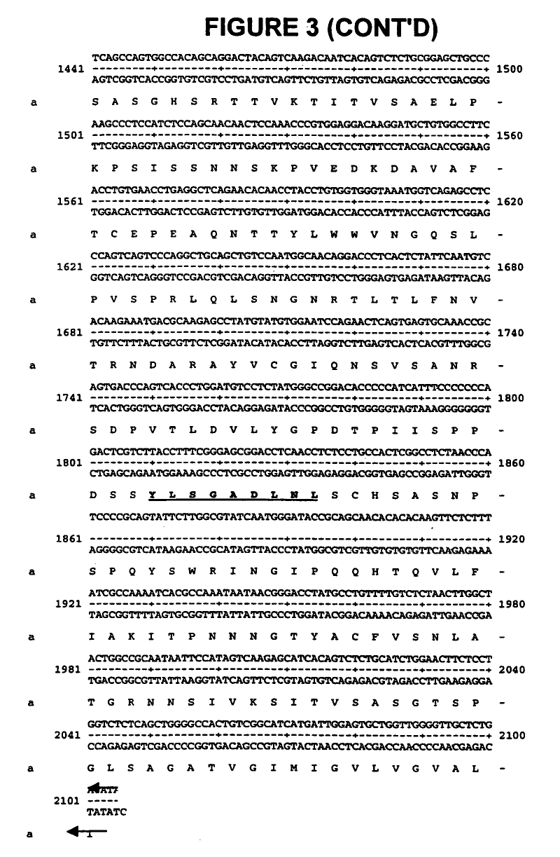

In another specific embodiment, the tumor-associated antigen is carcinoembryonic antigen CEA, a modified CEA or a fragment thereof. The sequence of native CEA is known in the art. The sequence of a modified CEA is shown in FIG. 3 or SEQ.ID.NO.:3 and SEQ.ID.NO.:4.

As noted above, the invention also encompasses administering nucleic acids coding for the antigen and/or the inducing agent. Accordingly, in one embodiment the antigen is administered as a nucleic acid sequence encoding a native gp100 protein or encoding a modified gp100 protein having the amino acid sequence shown in FIG. 2 or SEQ.ID.NO.: 2. The nucleic acid sequence may preferably have the sequence shown in FIG. 1 or SEQ.ID.NO.: 1. In another embodiment, the antigen is administered as a nucleic acid sequence encoding a native CEA antigen or a modified CEA antigen having the amino acid sequence shown in FIG. 3 or SEQ.ID.NO.: 4. The nucleic acid sequence may preferably have the sequence shown in FIG. 3 or SEQ.ID.NO.: 3. In one embodiment, the nucleic acid may be administered as free or naked DNA or RNA. In a preferred embodiment, the nucleic acid sequence is contained in a vector or plasmid. In one embodiment, the vectors of the invention may be viral such as poxvirus, adenovirus or alphavirus. Preferably the viral vector is incapable of integration in recipient animal cells. The elements for expression from said vector may include a promoter suitable for expression in recipient animal cells.

An example of an adenovirus vector, as well as a method for constructing an adenovirus vector capable of expressing an immunogen is described in U.S. Pat. No. 4,920,209 (incorporated herein by reference). Poxvirus vectors that can be used include, for example, vaccinia and canary pox virus (as described in U.S. Pat. Nos. 5,364,773, 4,603,112, 5,762,938, 5,378,457, 5,494,807, 5,505,941, 5,756,103, 5,833,975 and 5,990,091—all of which are herein incorporated by reference). Poxvirus vectors capable of expressing a nucleic acid of the invention can be obtained by homologous recombination as is known to one skilled in the art so that the polynucleotide of the invention is inserted in the viral genome under appropriate conditions for expression in mammalian cells (as described below).

In one preferred aspect the poxvirus vector is ALVAC (1) or ALVAC (2) (both of which have been derived from canarypox virus). ALVAC (1) (or ALVAC (2)) does not productively replicate in non-avian hosts, a characteristic thought to improve its safety profile. ALVAC (1) is an attenuated canarypox virus-based vector that was a plaque-cloned derivative of the licensed canarypox vaccine, Kanapox (Tartaglia et al., Virology 188:217-232 (1992); U.S. Pat. Nos. 5,505,941, 5,756,103 and 5,833,975—all of which are incorporated herein by reference). ALVAC (1) has some general properties which are the same as some general properties of Kanapox. ALVAC-based recombinant viruses expressing extrinsic antigens have also be n demonstrated efficacious as vaccine vectors (Tartaglia et al, In AIDS Research Reviews (vol. 3) Koff W., Wong-Staol F. and Kenedy R. C. (eds.), Marcel Dekker NY, pp. 361-378 (1993a); Tartaglia, J. et al., J. Virol. 67:2370-2375 (1993b)). For instance, mice immunized with an ALVAC (1) recombinant expressing the rabies virus glycoprotein were protected from lethal challenge with rabies virus (Tartaglia, J. et al., (1992) supra) demonstrating the potential for ALVAC (1) as a vaccine vector. ALVAC-based recombinants have also proven efficacious in dogs challenged with canine distemper virus (Taylor, J. et al., Virology 187:321-328 (1992)) and rabies virus (Perkus, M. E. et al., In Combined Vaccines and Simultaneous Administration: Current Issues and Perspective, Annals of the New York Academy of Sciences (1994)), in cats challenged with feline leukemia virus (Tartaglia, J. et al., (1993b) supra), and in horses challenged with equine influenza virus (Taylor, J. et al., In Proceedings of the Third International Symposium on Avian Influenza, Univ. of Wisconsin-Madison, Madison, Wis., pp. 331-335 (1993)).

ALVAC (2) is a second-generation ALVAC vector in which vaccinia transcription elements E3L and K3L have been inserted within the C6 locus (U.S. Pat. No. 5,990,091, incorporated herein by reference). The E3L encod s a protein capable of specifically binding to dsRNA. The K3L ORF has significant homology to El F-2. Within ALVAC (2) the E3L gene is under the transcriptional control of its natural promoter, whereas K3L has been placed under the control of the early/late vaccine H6 promoter. The E3L and K3L genes act to inhibit PKR activity in cells infected with ALVAC (II), allowing enhancement of the level and persistence of foreign gene expression.

Additional viral vector systems involve the use of naturally host-restricted poxviruses. Fowlpox virus (FPV) is the prototypic virus of the Avipox genus of the Poxvirus family. Replication of the avipox viruses is limited to avian species (Matthews, R. E. F., Intervirology, 17:42-44 (1982)) and there are no reports in the literature of avipox virus causing a productive infection in any non-avian species including man. This host restriction provides an inherent safety barrier to transmission of the virus to other species and makes use of avipox virus based vectors in veterinary and human applications an attractive proposition.

FPV has been used advantageously as a vector expressing immunogens from poultry pathogens. The hemagglutinin protein of a virulent avian influenza virus was expressed in an FPV recombinant. After inoculation of the recombinant into chickens and turkeys, an immune response was induced which was protective against either a homologous or a heterologous virulent influenza virus challenge (Taylor, J. et al,. Vaccine 6: 504-508 (1988)). FPV recombinants expressing the surface glycoproteins of Newcastle Disease Virus have also been developed (Taylor, J. et al., J. Virol. 64:1441-1450 (1990); Edbauer, C. et al., Virology 179:901-904 (1990); U.S. Pat. No. 5,766,599—incorporated herein by reference).

A highly attenuated strain of vaccinia, designated MVA, have also been used as a vector for poxvirus-based vaccines. Use of MVA is described in U.S. Pat. No. 5,185,146.

Other attenuated poxvirus vectors have been prepared by genetic modifications of wild type strains of virus. The NYVAC vector, for example, is derived by deletion of specific virulence and host-range genes from the Copenhagen strain of vaccinia (Tartaglia, J. et al. (1992), supra; U.S. Pat. Nos. 5,364,773 and 5,494,807—incorporat d herein by reference) and has proven useful as a recombinant vector in eliciting a protective immune response against an expressed foreign antigen.

Recombinant poxviruses can be constructed in two steps known in the art and analogous to the methods for creating synthetic recombinants of poxviruses such as the vaccinia virus and avipox virus (described in U.S. Pat. Nos. 4,769,330; 4,722,848; 4,603,112; 5,110,587; and 5,174,993—all of which are incorporated herein by reference).

Bacterial DNA useful in embodiments of the invention have be n disclosed in the art. These include, for example, Shigella, Salmonella, Vibrio cholerae, Lactobacillus, Bacille Calmette Guérin (BCG), and Streptococcus.

Non-toxicogenic Vibrio cholerae mutant strains that are also useful as bacterial vectors in embodiments of this invention are described, for exampl, in U.S. Pat. No. 4,882,278 (disclosing a strain in which a substantial amount of the coding sequence of each of the two ctxA alleles has been deleted so that no functional cholerae toxin is produced); WO 92/11354 (strain in which the irgA locus is inactivated by mutation; this mutation can be combined in a single strain with ctxA mutations); and WO 94/1533 (deletion mutant lacking functional ctxA and attRS1 DNA sequences). These strains can be genetically engineered to express heterologous antigens, as described in WO 94/19482. (All of the aforementioned issued patent/patent applications are incorporated herein by reference.) An effective immunogen dose of a Vibrio cholerae strain capable of expressing a polypeptide or polypeptide derivative encoded by a DNA molecule of the invention can contain, for example, about 1×10⁵ to about 1×10⁹, preferably about 1×10⁶ to about 1×10⁸ viable bacteria in an appropriate volume for the selected route of administration. Preferred routes of administration include all mucosal routes; most preferably, these vectors are administered intranasally or orally.

Attenuated Salmonella typhimurium strains, genetically engineered for recombinant expression of heterologous antigens or not, and their use as oral immunogens are described, for example, in WO 92/11361. Preferred routes of administration include all mucosal routes; most pr ferably, these vectors are administered intranasally or orally.

As will be readily appreciat d by those skill d in the art, oth r bacterial strains useful as vectors in embodiments of this invention include Shigella flexneri, Streptococcus gordonii, and Bacille Calmette Guerin (as described in WO 88/6626, WO 90/0594, WO 91/13157, WO 92/1796, and WO 92/21376; all of which are incorporated herein by reference). In bacterial vector embodiments of this invention, a polynucleotide of the invention may be inserted into the bacterial genome, can remain in a free state, or be carried on a plasmid.

In another embodiment of the invention, plasmids and/or free/naked DNA and RNA coding for the antigen can also be administered to an animal for immunogenic purposes (for example, U.S. Pat. No. 5,589,466; McDonnell and Askari, NEJM 334:42-45 (1996); Kowalczyk and Ertl, Cell Mol. Life Sci. 55:751-770 (1999)). Typically, this nucleic acid is a form that is unable to replicate in the target animal's cell and unable to integrate in said animal's genome. The DNA/RNA molecule is also typically placed under the control of a promoter suitable for expression in the animal's cell. The promoter can function ubiquitously or tissue-specifically. Examples of non-tissue specific promoters include the early Cytomegalovirus (CMV) promoter (described in U.S. Pat. No. 4,168,062) and the Rous Sarcoma Virus promoter. The desmin promoter is tissue-specific and drives expression in muscle cells. More generally, useful vectors have been described (i.e., WO 94/21797).

For administration of nucleic acids coding for antigen, said nucleic acids can encode a precursor or mature form of the antigen. When it encod s a precursor form, the precursor form can be homologous or heterologous. In the latter case, a eucaryotic leader sequence can be used, such as the leader sequence of the tissue-type plasminogen factor (tPA).

Standard techniques of molecular biology for preparing and purifying nucleic acids can be used in the preparation of aspects of the invention. For use as a source of an antigen, a nucleic acid of the invention can be formulated according to various methods known to those who are skilled in the art.

First, a nucleic acid can be used in a naked/free form, free of any delivery vehicles (such as anionic liposomes, cationic lipids, microparticles, (e.g., gold microparticles), precipitating agents (e.g., calcium phosphate)) or any other transfection-facilitating agent. In this case the nucleic acid can be simply diluted in a physiologically acceptable solution (such as sterile saline or sterile buffered saline) with or without a carrier. When present, the carrier preferably is isotonic, hypotonic, or weakly hypertonic, and has a relatively low ionic strength (such as provided by a sucrose solution (e.g., a solution containing 20% sucrose)).

Alternatively, a nucleic acid can be associated with agents that assist in cellular uptake. It can be, i.a., (i) complemented with a chemical agent that modifies the cellular permeability (such as bupivacaine; see, for example, WO 94/16737), (ii) encapsulated into liposomes, or (iii) associated with cationic lipids or silica, gold, or tungsten microparticles.

Cationic lipids are well known in the art and are commonly used for gene delivery. Such lipids include Lipofectin(also known as DOTMA (N-[1-(2,3-dioleyloxy)propyl]-N,N,N-trimethylammonium chloride), DOTAP (1,2-bis(oleyloxy)-3-(trimethylammonio) propane). DDAB (dimethyldioctadecylammonium bromide), DOGS (dioctadecylamidologlycyl spermine) and cholesterol derivatives such as DC-Chol (3 beta-(N-(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine), as, for example, described in WO 90/11092.

Other transfection-facilitating compounds can be added to a formulation containing cationic liposomes. A number of them are described in, for example, WO 93/18759, WO 93/19768, WO 94/25608, and WO 95/2397. They include, i.e., spermine derivatives useful for facilitating the transport of DNA through the nuclear membrane (see, for example, WO 93/18759) and membrane-p rmeabilizing compounds such as GALA, Gramicidin S, and cationic bile salts (see, for example, WO 93/19768).

Gold or tungsten microparticles can also be used for gene delivery (as described in WO 91/359 and WO 93/17706). In this case, the microparticle-coated polynucleotides can be injected via intradermal or intraepidermal routes using a needleless injection device (“gene gun”), such as those described, for example, in U.S. Pat. No. 4,945,050, U.S. Pat. No. 5,015,580, and WO 94/24263.

Anionic and neutral liposomes are also well-known in the art (see, for example, Liposomes: A Practical Approach, RPC New Ed, IRL Press (1990), for a detailed description of methods for making liposomes) and are useful for delivering a large range of products, including polynucleotides.

The amount of plasmid, naked/free DNA or RNA coding for an antigen to be administered to an animal generally depends on the strength of the promoter used in the DNA construct, the immunogenicity of the expressed gene product, the condition of the animal intended for administration (i.e. the weight, age, and general health of the animal), the mode of administration, and the type of formulation. In general, a therapeutically or prophylactically effective dose from about 1 μg to about 1 mg, preferably, from about 10 μg to about 800 μg and, more preferably, from about 25 μg to about 250 μg, can be administered to human adults. The administration can be achieved in a single dose, repeated at intervals, or incorporated into prime-boost protocols (as described below).

A nucleic acid encompassed by the invention can express one or several antigens. In addition, it can also express a cytokine (for example, such as interleukin-2 (IL-2), interleukin-12 (IL-12), granulocyte-macrophage colony stimulating factor (GM-CSF)) and or co-stimulatory molecules (for example, such as the B7 family of molecules) and/or other lymphokines that enhance the immune response. Thus, for example, a nucleic acid can include an additional DNA sequence encoding, for example, at least one additional tumor associated antigen (and/or immunogenic fragment, homolog, mutant or derivative thereof) and a cytokine and/or lymphokine and/or co-stimulatory mol cule plac d under the control of suitabl elements required for expression in an animal cell. Alternatively, embodiments of the invention may include several nucleic acids, each being capable of expressing an immunogen of the invention.

In additional embodiments of the invention, the antigen per se (or several antigens) can also be mixed with a cytokine and/or lymphokine and/or co-stimulatory molecule, and/or nucleic acids coding therefor.

An animal may be immunized with an antigen (or a nucleic acid coding therefor) by any conventional route, as is known to one skilled in the art. This may include, for example, immunization via a mucosal (e.g., ocular, intranasal, oral, gastric, pulmonary, intestinal, rectal, vaginal, or urinary tract) surface, via the parenteral (e.g., subcutaneous, intradermal, intramuscular, intravenous, or intraperitoneal) route or intranodally. Preferred routes depend upon the choice of the antigen and/or nucleic acid employed. The administration can be achieved in a single dose or repeated at intervals. The appropriate dosage depends on various parameters understood by skilled artisans such as the immunogen itself, the route of administration and the condition of the animal to be vaccinated (weight, age and the like).

In one embodiment, the administration of the inducing agent and the antigen (i.e. step (b) of the method) may occur anywhere from about 2 to 8 weeks, preferably 3 to 6 weeks following the initial pre-priming with the inducing agent (i.e. step (a) of the method). Most preferably, step (b) occurs from about 3 to 4 weeks after step (a).

The dose of the inducing agent is preferably from about 1 to about 50 limit of flocculation units (Lfu), more preferably 4-10 Lfu. The dose of the antigen is preferably from about 10 μg/mg bodyweight to about 1 μg/mg bodyweight, more preferably from about 50 μg/mg to about 500 μg/mg. When the antigen is administered as a nucleic acid sequence in a recombinant viral vector it is preferably in an amount from about 10⁶ to about 10⁹ pfu/ml, more preferably 5×10⁶ to about 5×10⁸ pfu/ml.

In one embodiment of the inv ntion, the antigen is a tumor antigen and the method can be used for the treatment of cancer. Accordingly, the present invention provides a method of tr ating or pr venting cancer in an animal comprising (a) administering an effective amount of inducing agent to the animal followed by (b) administering an effective amount of the inducing agent and a tumor antigen to the animal. Preferably, the tumor antigen is administered as a nucleic acid sequence encoding the tumor antigen.

The immunization of an animal with the tumor antigen (or nucleic acid coding therefor) in a cancer treatment of the invention may be for either a prophylactic or therapeutic purpose. When provided prophylactically, the tumor antigen (or nucleic acid coding therefor) is provided in advance of any evidence or in advance of any symptom due to cancer, or in patients rendered free of disease by conventional therapies but at significant risk for reoccurrence. The prophylactic administration of the tumor antigen (or nucleic acid coding therefor) serves to prevent or attenuate cancer in an animal. When provided therapeutically, the tumor antigen (or nucleic acid coding therefor) is provided at (or after) the onset of the disease or at the onset of any symptom of the disease. The therapeutic administration of the tumor antigen (or nucleic acid coding therefor) serves to attenuate the disease.

A particularly preferred method of immunizing an animal with the antigen (or nucleic acid coding therefor) encompasses a prime-boost protocol.

Recent studies have indicated that this protocol (i.e. prime-boost) is quite effective. Typically, an initial administration of an antigen or immunogen (or nucleic acid coding therefor) followed by a boost utilizing the antigen or a fragment thereof (or alternatively, a nucleic acid coding therefor) will elicit an enhanced immune response relative to the response observed following administration of either antigen (or nucleic acid coding therefor) or boosting agent. An example of a prime-boost methodology/protocol is described in WO 98/58956, which is incorporated herein by reference.

Accordingly, in another embodiment the present invention provides a method of enhancing an immune response to an antigen in an animal comprising (a) administering an inducing agent to the animal followed by (b) administering a first dose of the inducing agent and the antigen to the animal followed by (c) administering a second dose of the inducing agent and the antigen to th animal. Preferably, the second dose of the inducing agent and the antigen is administered anywhere from about 2 to about 8 weeks, preferably 3 to 6 weeks after the first dose administered in step (b).

Immunogenicity can be significantly improved if the antigens (or nucleic acids coding therefor) are, regardless of administration format (i.e. poxvirus, naked/free DNA, protein/peptide), co-immunized with adjuvants. Commonly, adjuvants are used as an 0.05 to 1.0 percent solution in phosphate-buffered saline. Adjuvants enhance the immunogenicity of an antigen but are not necessarily immunogenic themselves. Adjuvants may act by retaining the immunogen locally near the site of administration to produce a depot effect facilitating a slow, sustained release of antigen to cells of the immune system. Adjuvants can also attract cells of the immune system to an immunogen depot and stimulate such cells to elicit immune responses.

Adjuvants (including the use of immunostimulatory agents as adjuvants) have been used for many years to improve the host immune responses to, for example, vaccines. Intrinsic adjuvants, such as lipopolysaccharides, normally are the components of killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Some of these adjuvants are toxic, however, and can cause undesirable side-effects making them unsuitable for use in humans and many animals. Indeed, only aluminum hydroxide and aluminum phosphate (collectively commonly referred to as alum) are routinely used as adjuvants in human and veterinary vaccin s. The efficacy of alum in increasing antibody responses to diphtheria and tetanus toxoids is well established. Notwithstanding, it does have limitations. For example, alum is ineffective for influenza vaccination and inconsistently elicits a cell mediated immune response with other immunogens. The antibodies elicited by alum-adjuvanted antigens are mainly of the IgG1 isotype in the mouse, which may not be optimal for protection by some vaccinal agents.

A wide range of extrinsic adjuvants can provoke potent immune responses to antigens. These include saponins complexed to membrane protein antigens (immune stimulating complexes), pluronic polymers with mineral oil, killed mycobacteria and mineral oil, Freund's complete adjuvant, bacterial products such as muramyl dipeptide (MDP) and lipopolysaccharide (LPS), as well as lipid A, and liposomes.

To efficiently induce humoral immune responses (HIR) and cell-mediated immunity (CMI), antigens are often emulsified in adjuvants. Many adjuvants are toxic, inducing granulomas, acute and chronic inflammations (Freund's complete adjuvant, FCA), cytolysis (saponins and pluronic polymers) and pyrogenicity, arthritis and anterior uveitis (LPS and MDP). Although FCA is an excellent adjuvant and widely used in research, it is not licensed for use in human or veterinary vaccines because of its toxicity.

Desirable characteristics of ideal adjuvants include:

1) lack of toxicity;

2) ability to stimulate a long-lasting immune response;

3) simplicity of manufacture and stability in long-term storage;

4) ability to elicit both CMI and HIR to antigens administered by various routes, if required;

5) synergy with other adjuvants;

6) capability of selectively interacting with populations of antigen presenting cells (APC);

7) ability to specifically elicit appropriate T_{H}1 or T_{H}2 cell-specific immune responses; and

8) ability to selectively increase appropriate antibody isotype levels (for example, IgA) against antigens/immunogens.

U.S. Pat. No. 4,855,283 granted to Lockhoff et al on Aug. 8, 1989, which is incorporated herein by reference thereto, teaches glycolipid analogues including N-glycosylamides, N-glycosylureas and N-glycosylcarbamates, each of which is substituted in the sugar residue by an amino acid, as immuno-modulators or adjuvants. Thus, Lockhoff t al. (Chem. Int. Ed. Engl. 30:1611-1620 (1991)) reported that N-glycolipid analogs displaying structural similarities to the naturally-occurring glycolipids, such as glycophospholipids and glycoglycerolipids, are capable of eliciting strong immune responses in both herpes simplex virus vaccine and pseudorabies virus vaccine. Some glycolipids have been synthesized (from long chain-alkylamines and fatty acids that are linked directly with the sugars through the anomeric carbon atom) to mimic the functions of the naturally occurring lipid residues.

U.S. Pat. No. 4,258,029 granted to Moloney and incorporated herein by reference thereto, teaches that octadecyl tyrosine hydrochloride (OTH) functions as an adjuvant when complexed with tetanus toxoid and formalin inactivated type I, II and III poliomyelitis virus vaccine. Nixon-George et al. (J. Immunol. 14:4798-4802 (1990)) have also reported that octadecyl esters of aromatic amino acids complexed with a recombinant hepatitis B surface antigen enhanced the host immune responses against hepatitis B virus.

Adjuvant compounds may also be chosen from the polymers of acrylic or methacrylic acid and the copolymers of maleic anhydride and alkenyl derivative. Adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Preferably, a solution of adjuvant according to the invention, especially of carbomer, is prepared in distilled water, preferably in the presence of sodium chloride, the solution obtained being at acidic pH. This stock solution is diluted by adding it to the desired quantity (for obtaining the desired final concentration), or a substantial part thereof, of water charged with NaCl, preferably physiological saline (NaCL 9 g/l) all at once in several portions with concomitant or subsequent neutralization (pH 7.3 to 7.4), preferably with NaOH. This solution at physiological pH will be used as it is for mixing with the vaccine, which may be especially stored in freeze-dried, liquid or frozen form. The polymer concentration in the final vaccine composition will be 0.01% to 2% w/v, more particularly 0.06 to 1% w/v, preferably 0.1 to 0.6% w/v.

P rsons skilled in the art can also refer to U.S. Pat. No. 2,909,462 (incorporated herein by reference) which describes such acrylic polymers cross-linked with a polyhydroxylated compound having at least 3 hydroxyl groups (preferably not more than 8), the hydrogen atoms of the at least three hydroxyls being replaced by unsaturated aliphatic radicals having at least 2 carbon atoms. The preferred radicals are those containing from 2 to 4 carbon atoms (e.g. vinyls, allyls and other ethylenically unsaturated groups). The unsaturated radicals may themselves contain other substituents, such as methyl. The products sold under the name Carbopol (BF Goodrich, Ohio, USA) are particularly appropriate. They are cross-linked with allyl sucrose or with allyl pentaerythritol. Among them, there may be mentioned Carbopol (for example, 974P, 934P and 971P). Among the copolymers of maleic anhydride and alkenyl derivative, the copolymers EMA (Monsanto; which are copolymers of maleic anhydride and ethylene, linear or cross-linked, (for example cross-linked with divinyl ether)) are preferred. Reference may be made to J. Fields et al. (Nature, 1960, 186: 778-780) for a further description of these chemicals (incorporated (herein by reference).

In one aspect of this invention, adjuvants useful in any of the embodiments of the invention described herein are as follows. Adjuvants for parenteral immunization include aluminum compounds (such as aluminum hydroxide, aluminum phosphate, and aluminum hydroxy phosphate). The antigen can be precipitated with, or adsorbed onto, the aluminum compound according to standard protocols. Other adjuvants such as RIBI (ImmunoChem, Hamilton, Mont.) can also be used in parenteral administration.

Adjuvants for mucosal immunization include bacterial toxins (e.g., the cholera toxin (CT), the E. coli heat-labile toxin (LT), the Clostridium difficile toxin A and the pertussis toxin (PT), or combinations, subunits, toxoids, or mutants thereof). For example, a purified preparation of native cholera toxin subunit B (CTB) can be of use. Fragments, homologs, derivatives, and fusion to any of these toxins are also suitable, provided that they retain adjuvant activity. Preferably, a mutant having reduced toxicity is used. Suitable mutants have been described (e.g., in WO 95/17211 (Arg-7-Lys CT mutant), WO 96/6627 (Arg-192-Gly LT mutant), and WO 95/34323 (Arg-9-Lys and Glu-129-Gly PT mutant)). Additional LT mutants that can be used in the methods and compositions of the invention include, for example Ser-63-Lys, Ala-69-Gly, Glu-110-Asp, and Glu-112-Asp mutants. Other adjuvants (such as a bacterial monophosphoryl lipid A (MPLA) of various sources (e.g., E. coli, Salmonella minnesota, Salmonella typhimurium, or Shigella flexneri, saponins, or polylactide glycolide (PLGA) microspheres) can also be used in mucosal administration.

Adjuvants useful for both mucosal and parenteral immunization include polyphosphazene (for example, WO 95/2415), DC-chol (3 b-(N-(N′,N′-dimethyl aminomethane)-carbamoyl) cholesterol (for example, U.S. Pat. No. 5,283,185 and WO 96/14831) and QS-21 (for example, WO 88/9336).

Antigens and inducing agents (or nucleic acids coding therefor) encompassed by embodiments of the invention may be formulated into pharmaceutical compositions in a biologically compatible form suitable for in vivo animal immunization. By “biologically compatible form suitable for in vivo animal immunization” is meant a form of the substance to be administered in which any toxic effects are outweighed by the therapeutic effects. The substances may be administered to animals in need thereof. Immunization with a therapeutically active amount of the pharmaceutical compositions of the present invention, or an “effective amount”, are defined as an amount effective, at dosages and for periods of time necessary to achieve the desired result of enhancing an animal's immune response to the antigen. A therapeutically effective amount of a substance may vary according to factors such as the disease state, age, sex, and weight of the animal, and the ability of immunogen to elicit a desired response in the animal. Dosage regima may be adjusted to provide the optimum therapeutic response. For example, several divided doses may be administered daily or the dose may be proportionally reduced as indicated by the exigencies of the immunization context.

Additionally, the antigens and inducing agents (or nucleic acids therefor) and inducing agents may be in admixture with a suitable carrier, dilu nt, or excipi nt such as sterile water, physiological saline, glucose or the like to form suitable pharmaceutical compositions. The compositions can also be lyophilized. The compositions may also contain auxiliary substances such as wetting or emulsifying agents, pH buffering agents, adjuvants, gelling or viscosity enhancing additives, preservatives, flavoring agents, colors, and the like, depending upon the route of administration and the preparation desired.

Accordingly, the present invention provides a vaccine composition comprising an inducing agent and an antigen in admixture with a pharmaceutically acceptable diluent or carrier. The inducing agent and/or the antigen may be in the form of a protein or a nucleic acid encoding the protein. In one embodiment, the vaccine composition comprises a recombinant fusion protein comprising an inducing agent linked to an antigen. In another embodiment, the vaccine composition comprises a chimeric nucleic acid sequence comprising a first nucleic acid sequence encoding an inducing agent linked to a second nucleic acid sequence encoding an antigen.

The present invention also includes a use of a vaccine composition of the present invention to enhance an immune response as well as a use of a vaccine composition of the present invention to prepare a medicament to enhance or immune response.

Animals may be immunized with the pharmaceutical compositions via a number of convenient routes, such as by injection (intradermal, intramuscular, subcutaneous, intravenous, intranodal etc.), or by oral administration, inhalation, transdermal application, or rectal administration, or any other route of immunization that enables the modulation of an animal's immune system. Depending on the route of immunization, the pharmaceutical composition may be coated in a material to protect the compound from the action of enzymes, acids and other natural conditions which may inactivate the compound.

The compositions described herein can be prepared by per se known methods for the preparation of pharmaceutically acceptable compositions with which animals can be immunized, such that an effective quantity of th antigen and inducing agent (or nucleic acid coding therefor) is combined in a mixture with a pharmaceutically acceptable vehicle (for example, diluent and/or carrier). Suitable vehicles are described, for xample, in Remington's Pharmaceutical Sciences (Remington's Pharmaceutical Sci nces (1985), Mack Publishing Company, Easton, Pa., USA). On this basis, the pharmaceutical compositions include, albeit not exclusively, solutions of the substances in association with one or more pharmaceutically acceptable carriers and/or diluents, and may be contained in buffered solutions with a suitable pH and/or be iso-osmotic with physiological fluids. In this regard, reference can be made to U.S. Pat. No. 5,843,456. Reference can also be made to the textbook Vaccine Design: the Subunit and Adjuvant Approach, Michael F. Powell and Mark J. Newman, eds. Plenum Press, New York, 1995.

The following non-limiting examples are illustrative of the present invention:

### EXAMPLES

### Example 1

### Enhancement of an Immune Response to Gp100 Antigens Using TT as an Inducing Agent

Summary

The A2Kb transgenic mouse was used to assess the immunogenicity of recombinant ALVAC vectors expressing the native gp100 gene and/or the modified gp100 gene (FIG. 1 or SEQ.ID.NO.:1). HLA-A0201-restricted gp100-specific CTL (cytotoxic T cell) responses were assessed. The modifi d gp100 insert used to construct the ALVAC recombinants contained 2 point mutations, one at position 210 where threonine (T) of the native gp100 was replaced by methionine (M), and the other at position 288 where the native alanine (A) was replaced by valine (V) (as described in U.S. patent application Ser. No. 09/693,755, filed Oct. 20, 2000—incorporated herein by reference. See also FIG. 2 and SEQ.ID.NO.: 2). Mice were primed with vaccine quality tetanus toxoid (TT) in saline. The animals were then immunized and boosted with ALVAC recombinants in combination with TT. In parallel, control studi s involving mice unprimed with TT, and boosted with ALVAC recombinants in the presence or absence of TT were also examined for their capability to generate gp100-specific CTL respons s.

The analysis of the sp cificity of ALVAC recombinant-induced CTLs was focused on HLA-A0201-restricted human CTL epitopes gp100 (209-217) (i.e. amino acid sequence ITDQVPFSV, SEQ.ID.NO.:5) and gp100 (280-288) (i.e. amino acid sequence YLEPGPVTA, SEQ.ID.NO.:6) of the native gp100 molecule. For the transgenic mice that received the modified gp100 ALVAC recombinant vectors, effector responses directed against the mutated counterparts of these epitopes were examined (namely, gp100 (209M) (i.e. amino acid sequence IMDQVPFSV, SEQ.ID.NO.:7) and gp100 (280M) (i.e. amino acid sequence YLEPGPVTV, SEQ.ID.NO.:8)).

Materials and Methods

Methods of the peptide synthesis, cell culture and cytotoxic T cell (CTL) assay were conducted via well documented and standard methodologies, and as such are well within the scope of those skilled in the art.

Vectors

Recombinant ALVAC vectors were constructed via methodologies and/or processes well known to those skilled in the art.

ALVAC (1) parent vector is described in U.S. Pat. Nos. 5,505,941, 5,756,103, 5,833,975—all of which are incorporated herein by reference. ALVAC (2) parent vector is described in U.S. Pat. No. 5,990,091, which is incorporated herein by reference. Modified gp100 is described in U.S. patent application Ser. No. 09/693,755, filed on Oct. 20, 2000—which is incorporated herein by reference.

Synthesis of Peptides

Solid phase peptide syntheses were conducted on an ABI 430A automated peptide synthesizer according to the manufacturer's standard protocols. The peptides were cleaved from the solid support by treatment with liquid hydrogen fluoride in a presence of thiocresole, anisole, and methyl sulfide. The crude products were extracted with trifluoroacetic acid (TFA) and precipitated with diethyl ether. All peptides were stored in lyophilized form at −20° C.

The peptid s synthesized were:

CLP 168-ITDQVPFSV (SEQ.ID.NO.:5)

CLP 169-YLEPGPVTA (SEQ.ID.NO.:6)

CLP 572-IMDQVPFSV (SEQ.ID.NO.:7)

CLP 573-YLEPGPVTV (SEQ.ID.NO.:8)

CTL Assay

Mice of the B1O background (transgenic for the A2Kb chimeric gene) were purchased from the Scripps Clinic in California, USA. For tetanus toxoid (TT) priming, 20.0 μg of Aventis Pasteur's TT vaccine prepared in 100.0 μl of sterile phosphate buffered saline (PBS, pH 7.2) was injected into the quadriceps and gluteus muscles of each mouse. 4 weeks later, the animals were boosted with an inoculum of 100.0 μl of PBS (pH 7.2) containing 1×10⁷ plague-forming units (p.f.u.) of ALVAC recombinant with/without 20.0 μg of TT using the same intramuscular route. Mice were again boosted with the respective inoculum 25 days later. 11 to 35 days after the final injection, spleenocytes of the experimental mice were prepared and cultured to enrich for CTLs before being assayed for effector activity. In vitro re-stimulation of the in vivo generated CTLs was performed by co-culturing in a 25 cm² tissue culture flask 3×10⁷ responder cells (i.e., splenocytes) with 1.3×10⁷ irradiated autologous LPS (lipopolysaccharide)-blasts which had been pulsed with the appropriate peptide (100.0μ per 10⁸ cells). Cultures were kept in a 37° C., humidified CO₂ incubator for 7 days before being tested for effector function in a standard 5 hr in vitro ⁵¹Cr-release CTL assay as follows. The responders were harvested from the day 7 bulk cultures and washed twice with RPMI-1640 medium (without bovine serum). The positive target was created by incubating 3-5×10⁶ P815-A2Kb transfectant cells with 100.0μ of the specified peptide overnight in a 37° C. CO₂ incubator. The target cells were then labeled with ⁵¹Cr at 250.0 μCi per 1×10⁶ cells for 1 hr in the presence of 15.0μ of the same test peptides and 15.0μ of human β2-microglobulin. After washing twice with complete medium to remove excess free ⁵¹Cr, the targets were incubated at 2.5×10³ with different numbers of the responders for 5 hr in a 37° C. CO₂ incubator. Supernatant aliquots were removed and counted for radioactivity.

Results

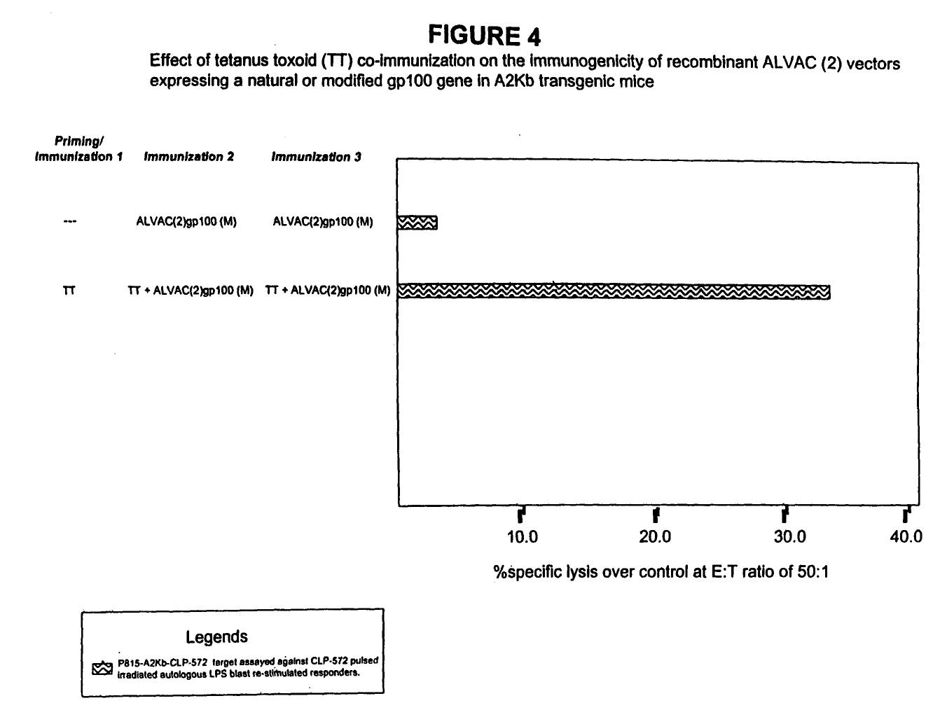

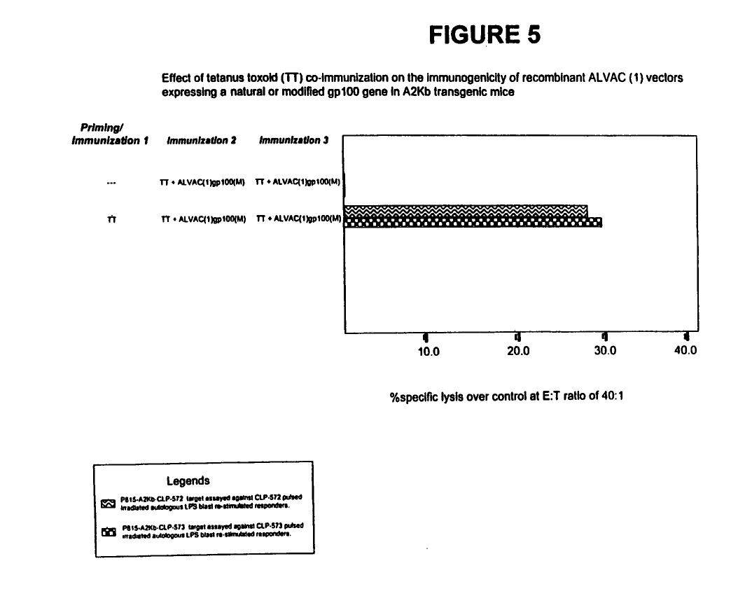

The results obtained for studies using ALVAC (2) and ALVAC (1) recombinants expressing modified gp100 are depicted in FIGS. 4 and 5, respectively. The results indicate that tetanus toxoid priming results in a clearly enhanced immune response to the immunogen modified gp100 when the vector encoding for the immunogen is administered as a mixture with tetanus toxoid. This was not vector specific since said enhancement was observed with both vectors utilized.

### Example 2

### Enhancement of an Immune Response to GP100 and CEA Antigens Using TT or DT as an Inducing Agent

Summary

The A2Kb transgenic mouse was used to assess the immunogenicity of recombinant ALVAC vectors expressing native gp100, modified gp100 or the full length carcinoembryonic antigen (CEA). HLA-A0201-restricted gp100 or CEA specific reactive T cell responses were assessed using ELISPOT assays. The preparation of the gp100 ALVAC recombinants are describ d in Example 1. The full length CEA gene was incorporated into the ALVAC vector. ALVAC gp100 and ALVAC CEA immunized mice were primed with vaccine quality diphtheria toxoid (DT) and tetanus toxoid (TT) in saline respectively. The animals were then immunized and boosted with ALVAC recombinants in combination with TT or DT. In parallel, control studies involving mice unprimed with TT, and boosted with ALVAC recombinants in the presence or absence of TT were also examined for their capability to generate gp100 or CEA specific T cell responses.

The analysis of the specificity of ALVAC gp100 recombinant-induced T cell reactivity was focused on HLA-A0201-restricted human CTL epitopes gp100 (209-217) (i.e. amino acid sequence ITDQVPFSV, SEQ.ID.NO.:5), (210M; i.e. amino acids sequence IMDQVPFSV, SEQ.ID.NO.:7), gp100 (280-288) (i.e. amino acid sequence YLEPGPVTA, SEQ.ID.NO.:9) and (288V; i.e. amino acid sequence YLEPGPVTV, SEQ.ID.NO.:8) of the native and modifi d gp100 molecule. For the CEA analysis focus was given to the HLA-A0201 peptides CAP-1 (i.e. amino acid s quence YLSGANLNL, SEQ.ID.NO.:10) and its modified CAP-6D (i.e. amino acid sequ nce (YLSGADLNL, SEQ.ID.NO.:11) of the native CEA.

Materials and Methods

Methods of the peptide synthesis, cell culture and ELISPOT assay were conducted via well documented and standard methodologies, and as such are well within the scope of those skilled in the art.

Vectors

Recombinant ALVAC vectors were constructed via methodologies and/or processes well known to those skilled in the art.

ALVAC (1) parent vector is described in U.S. Pat. Nos. 5,505,941, 5,756,103, 5,833,975—all of which are incorporated herein by reference. ALVAC (2) parent vector is described in U.S. Pat. No. 5,990,091, which is incorporated herein by reference. Modified gp100 is described in U.S. patent application Ser. No. 09/693,755, filed on Oct. 20, 2000—which is incorporated herein by reference.

Synthesis of Peptides

Solid phase peptide syntheses were conducted on an ABI 430A automated peptide synthesizer according to the manufacturer's standard protocols. The peptides were cleaved from the solid support by treatment with liquid hydrogen fluoride in a presence of thiocresole, anisole, and methyl sulfide. The crude products were extracted with trifluoroacetic acid (TFA) and precipitated with diethyl ether. All peptides were stored in lyophilized form at −20° C.

The peptides synthesized were:

CLP 168-ITDQVPFSV

CLP 169-YLEPGPVTA

CLP 572-IMDQVPFSV

CLP 573-YLEPGPVTV

CLP 165-YLSGANLNL

CLP 1510-YLSGADLNL

ELISPOT Assay

Mice of the B10 background (transgenic for the A2Kb chim ric gene) were purchased from the Scripps Clinic in California, USA. For tetanus toxoid (TT) and diphtheria toxoid priming, 20.0 μg of Pasteur Merieux Connaught's TT and/or DT vaccine was prepared in 100.0 μl of sterile phosphate buffered saline separately (PBS, pH 7.2) and was injected into the quadriceps and gluteus muscles of each mouse. Three weeks later, the animals were boosted with an inoculum of 100.0 μl of PBS (pH 7.2) containing 2×10⁷ plague-forming units (p.f.u.) of either ALVAC recombinant with/without 20.0 μg of TT or DT using the same intramuscular route. Mice were again boosted with the respective inoculum 21 days later. After the final injection, splenocytes of the experimental mice were prepared and cultured to enrich for either gp100 or CEA reactive T cells before being assayed for effector activity. In vitro re-stimulation of the in vivo generated T cells was performed by culturing in a 25 cm² tissue culture flask 1×10⁸ responder cells (i.e., splenocytes) with peptide (100.0 μg per 10⁸ cells). Cultures were kept in a 37° C., humidified CO₂ incubator for 7 days before being tested for effector function in a standard IFN gamma ELISPOT assay as follows. The responders were harvested from the day 7 bulk cultures and washed twice with AIM-V medium (without bovine serum). The target cells were generated by incubating 1×10⁶ P815-A2Kb transfectant cells with 10 μg of the specified peptide 3-5 hours in a 37° C. CO₂ incubator. The target cells were washed twice with complete medium to remove excess free peptide and plated on an ELISPOT plate at 1×10⁵ cells/well. Responding T cells were harvest d from the tissue culture flasks, washed with excess AIM-V medium and counted. The responding T cells were then co-cultured with the stimulators cells on the ELISPOT plate at 1×10⁵ responders/well.

Results

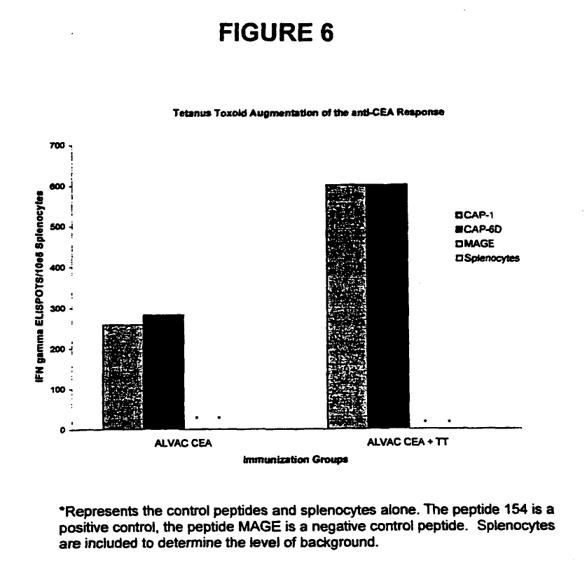

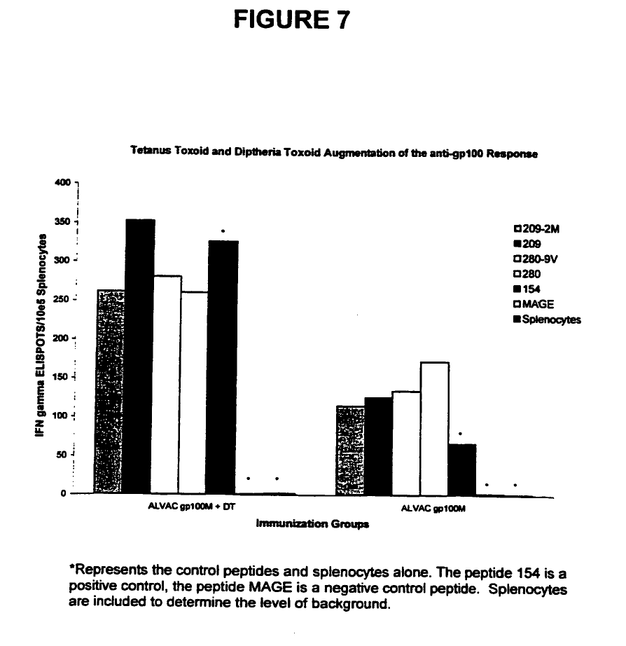

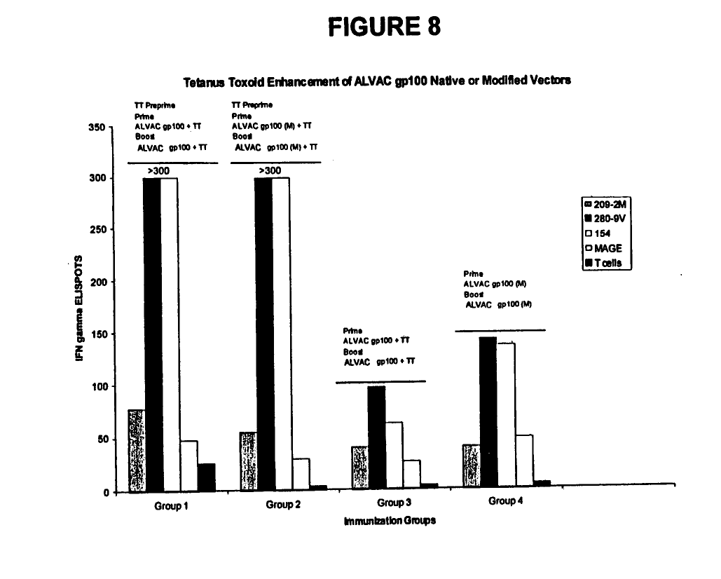

The results obtained for studies utilizing ALVAC CEA and ALVAC modified gp100 recombinants are shown in FIGS. 6 and 7, respectively. The results obtained for studies using native or modified gp100 are shown in FIG. 8. Th r suits indicate that diphtheria tox id and tetanus toxoid priming results in a clearly enhanced immun response to the modified gp100, gp100 and CEA antigens when the vector encoding the antigen is administered as a mixture with tetanus toxoid or diphtheria toxoid. This was not vector specific since said enhancement was observed with both vectors utilized.

Whereas the invention is susceptible to various modification and/or alternative forms, specific embodiments have been shown by way of example and are herein described in detail. However, it should be understood that it is not intended to limit the invention to the particular embodiments shown, but on the contrary, the invention is to cover all modification, equivalents, and/or alternatives falling within the spirit and scope of the invention as defined by the appended claims.

All publications, patents and patent application referred to herein, ar herein incorporated by reference in their entirety to the same extent as if ach individual publication, patent or patent application was specifically and individually indicated to be incorporated by reference in its entirety.

We claim:

1. A method of enhancing an immune response to an antigen in an animal comprising (a) administering an effective amount of an inducing agent to the animal followed by (b) administering an effective amount of the inducing agent and the antigen to the animal.

2. A method according to claim 1 wherein the inducing agent is a bacterial toxoid.

3. A method according to claim 2 wherein the bacterial toxoid is tetanus toxoid or diphtheria toxoid.

4. A method according to any one of claims 1 to 3 wherein the antigen is a protein.

5. A method according to claim 4 wherein the antigen is selected from the group consisting of tumor antigens, autoimmune antigens and an antigen isolated from a pathogenic organism.

6. A method according to claim 5 wherein the tumor antigen is selected from the group consisting of gp100, carcinoembryonic antigen, tyrosinase, TRP-1, TRP-2, MART-1/Melan A, MAGE family, BAGE family, GAGE family, RAGE family, KSA, NY ESO-1, MUC-1, MUC-2, p53, p185, HER2/neu, PSA and PSMA and modified forms thereof.

7. A method according to claim 5 wherein the tumor antigen is gp100 or carcinoembryonic antigen or a modified form thereof.

8. A method according to claim 7 wherein the antigen is GP100 or modified gp100 having the sequence as shown in FIG. 2 (SEQ.ID.NO.:2).

9. A method according to claim 7 wherein the antigen is carcinoembryonic antigen (CEA) or modified CEA having the sequence shown in FIG. 3 (SEQ.ID.NO.:4).

10. A method according to any one of claims 1-9 wherein the antigen is administered as a nucleic acid sequence encoding the antigen.

11. A method according to claim 10 wherein the nucleic acid sequence is in a vector, plasmid or bacterial DNA.

12. A method according to claim 11 wherein the vector is a viral vector.

13. A method according to claim 12 wherein the viral vector is selected from adenovirus, alphavirus, and poxvirus.

14. A method according to claim 13 wherein the poxvirus is selected from the group consisting of vaccinia, fowlpox and avipox.

15. A method of claim 14 wherein the poxvirus is selected from the group comprising TROVAC, ALVAC, NYVAC, and MVA.

16. A method according to any one of claims 1 to 15 wherein step (b) occurs from about 3 weeks to about 6 weeks after step (a).

17. A method according to any one of claims 1 to 15 wherein step (b) occurs from about 3 weeks to about 4 weeks after step (a).

18. A method according to any one of claims 1 to 17 further comprising (c) administering a second dose of the inducing agent and the antigen.

19. A method according to claim 18 wherein step (c) occurs from about 3 weeks to about 6 we ks after step (b).

20. A method according to claim 18 wherein step (c) occurs from about 3 weeks to about 4 weeks after step (b).

21. A method according to any one of claims 1-20 wherein the antigen is administered in combination with at least one member selected from the group consisting of cytokines, lymphokines, co-stimulatory molecules, and nucleic acids coding therefor.

22. A method according to any one of claims 1-21 wherein the antigen is administered in combination with an adjuvant.

23. A method according to any one of claims 1-22 wherein the inducing agent is tetanus toxoid or diphtheria toxoid and the antigen is a tumor antigen.

24. A method according to claim 23 for the treatment of cancer.

25. A vaccine composition comprising an inducing agent and an antigen.

26. A use of a vaccine composition according to claim 25 to enhanc an immune response.


SEQUENCE LISTING (11 sequences)

SEQ ID NO: 1 - DNA, 1986 bp, Artificial
  modified gp 100
atggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacacgatggaagtgactgtctaccatcgccggggatcccggagctatgtgcctcttgctcattccagctcagccttcaccattatggaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgttcaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaacagccccctcctcagtgggcagcaggtctga

SEQ ID NO: 2 - protein, 661 aa, Artificial
  modified gp 100
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTIMDQVPFSVSVSQLRALDGGNKHFLRNQPLTFALQLHDPSGYLAEADLSYTWDFGDSSGTLISRALVVTHTYLEPGPVTVQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 3 - DNA, 2106 bp, Artificial
  modified CEA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 4 - protein, 701 aa, Artificial
  modified CEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGADLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 5 - protein, 9 aa, Artificial
  Synthetic peptide, amino acids 209-217 of gp 100, referred to as CLP 168
ITDQVPFSV

SEQ ID NO: 6 - protein, 9 aa, Artificial
  Synthetic peptide, amino acids 280-288 of gp 100, referred to as CLP 169
YLEPGPVTA

SEQ ID NO: 7 - protein, 9 aa, Artificial
  Synthetic peptide, amino acids 209-217 of modified gp 100, referred to as CLP 572
IMDQVPFSV

SEQ ID NO: 8 - protein, 9 aa, Artificial
  Synthetic peptide, amino acids 280-288 of modified gp 100, referred to as CLP 573
YLEPGPVTV

SEQ ID NO: 9 - protein, 9 aa, Artificial
  Synthetic peptide, amino acids 280-288 of gp 100, referred to as CLP 169
YLEPGPVTA

SEQ ID NO: 10 - protein, 9 aa, Artificial
  Synthetic peptide, peptide of CEA, referred to as CAP-1
YLSGANLNL

SEQ ID NO: 11 - protein, 9 aa, Artificial
  Synthetic peptide, peptide of modifed CEA, referred to as CAP-6D
YLSGADLNL